(19) United States
(12) Patent Application Publication
(10) Pub. No.: US 2015/0126869 A1
(43) Pub. Date: May 7, 2015

(54) ULTRASOUND DIAGNOSTIC APPARATUS, CONTROLLER OF ULTRASOUND DIAGNOSTIC APPARATUS, AND CONTROL METHOD OF ULTRASOUND DIAGNOSTIC APPARATUS

(71) Applicant: KONICA MINOLTA INC., Tokyo (JP)

(72) Inventor: Akihiro KAWABATA, Tokyo (JP)

(73) Assignee: KONICA MINOLTA INC.

(21) Appl. No.: 14/534,066

(22) Filed: Nov. 5, 2014

(30) Foreign Application Priority Data
Nov. 6, 2013 (JP) 2013-230021

(51) Int. Cl. A61B 8/08; A61B 8/00; A61B 8/14

(52) U.S. Cl. CPC A61B 8/5246; A61B 8/14; A61B 8/488; A61B 8/5269; A61B 8/54; A61B 8/463
USPC 600/440

(57) ABSTRACT

An ultrasound diagnostic apparatus configured in a manner connectable to an ultrasound probe and an indicator includes: a transmission unit; a reception unit; a B mode image generation unit; a C mode image generation unit; and a display processing unit, wherein the C mode image generation unit compares the first C mode image data and second C mode image data generated before the first C mode image data, determines whether there is a noise part in the first C mode image data based on the comparison result, eliminates the noise part when there is the noise part in the first C mode image data, and generates a first C mode image based on the first C mode image data from which the noise part is eliminated.

The entire disclosure of Japanese Patent Application No. 2013-230021 filed on Nov. 6, 2013 including description, claims, drawings, and abstract are incorporated herein by reference in its entirety.

### BACKGROUND OF THE INVENTION

1. Field of the Invention

The present application relates to an ultrasound diagnostic apparatus, a controller of the ultrasound diagnostic apparatus, and a control method of the ultrasound diagnostic apparatus.

2. Description of the Related Art

Based on a reflected ultrasound acquired by transmitting/receiving an ultrasound to/from the inside of a subject through an ultrasound probe, an ultrasound diagnostic apparatus images information inside the subject as an ultrasound image. As the ultrasound image, there are a brightness (B) mode image and a color flow (C) mode image superimposed and displayed on the B mode image.

The B mode image is an internal tissue of the subject imaged by displaying amplitude strength of a reflected ultrasound in luminance.

On the other hand, the C mode image is bloodstream information displayed in color in a region of interest (hereinafter, referred to as “ROI”) specified in the B mode image. This C mode image is, for example, an image which is bloodstream information in the ROI displayed with bloodstream in a direction getting closer to an ultrasound probe being displayed in a red component and bloodstream in a direction getting farther therefrom being displayed in a blue component. The C mode image is superimposed and displayed on the B mode image.

When the C mode image is generated, there may be a case where information other than information related to a bloodstream component is included in an acquired reflected ultrasound and a noise component-derived image (hereinafter, referred to as “noise image”) appears in the displayed C mode image in addition to a bloodstream component-derived image. Thus, an ultrasound diagnostic apparatus which performs processing to calculate an average value, the maximum value, or a rate of change between predetermined frames based on information of C mode images of a plurality of image frames and which performs processing without displaying a noise image in the C mode image by threshold processing has been proposed (see, for example, JP 05-277111 A).

### SUMMARY OF THE INVENTION

In the above described related technique, technique to control generation of a noise image in a C mode image has been desired. An object of a non-limiting exemplary embodiment of the present application is to provide an ultrasound diagnostic apparatus, a controller of the ultrasound diagnostic apparatus, and a control method of the ultrasound diagnostic apparatus which are capable of controlling generation of a noise image in a C mode image more effectively.

To achieve the abovementioned object, according to an aspect of the present application, an ultrasound diagnostic apparatus configured in a manner connectable to an ultrasound probe and an indicator reflecting one aspect of the present invention comprises: a transmission unit configured to perform transmission processing to drive the ultrasound probe and to transmit an ultrasound to a subject; a reception unit configured to perform reception processing to generate a reception signal based on a reflected ultrasound which is from the subject and is received by the ultrasound probe; a B mode image generation unit configured to generate a B mode image based on the reception signal; a C mode image generation unit configured to generate a first C mode image based on the reception signal; and a display processing unit configured to generate a composite image in which the C mode image is superimposed and displayed on the B mode image and to output the composite image to the indicator, wherein the C mode image generation unit compares the first C mode image data and second C mode image data generated before the first C mode image data, determines whether there is a noise part in the first C mode image data based on the comparison result, eliminates the noise part when there is the noise part in the first C mode image data, and generates a first C mode image based on the first C mode image data from which the noise part is eliminated.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is an example of a functional block diagram of an ultrasound diagnostic apparatus according to a first embodiment of the present application;

FIG. 2 is an example of a configuration view of hardware of the ultrasound diagnostic apparatus according to the first embodiment of the present application;

FIG. 3 is an example of a detail functional block diagram of a noise cut unit of the ultrasound diagnostic apparatus according to the first embodiment of the present application;

FIG. 4 is an example of an operation flowchart of the ultrasound diagnostic apparatus according to the first embodiment of the present application; and

FIG. 5A and FIG. 5B are auxiliary views for describing a noise determination unit of the ultrasound diagnostic apparatus according to the first embodiment of the present application.

### DESCRIPTION OF THE PREFERRED EMBODIMENTS

Hereinafter, an embodiment of the present invention will be described with reference to the drawings. However, the scope of the invention is not limited to the illustrated examples.

Inventors of the present application have studied a characteristic of a method disclosed in JP 05-277111 A in detail. As a result, it is found that the technique disclosed in JP 05-277111 A cannot eliminate a noise image adequately. Specifically, since a noise (hereinafter, referred to as “clutter noise”) which appears due to movement of an ultrasound probe or the like includes signal strength at the same level with that of weak bloodstream, an influence of the clutter noise is not eliminated by the method disclosed in JP 05-277111 A. Accordingly, specifically in a case of diagnosing with an ultrasound image while moving an ultrasound probe, such as a case of an ultrasound image diagnosis of a finger or the like of a patient with rheumatoid arthritis, an influence of a clutter noise appears prominently in a displayed C mode image.

The inventors of the present application have earnestly studied technique to control generation of a noise image in a C mode image effectively even in such a case and have found a new ultrasound diagnostic apparatus, controller of the ultrasound diagnostic apparatus, and method of the ultrasound diagnostic apparatus. An outline of an ultrasound diagnostic apparatus, a controller of the ultrasound diagnostic apparatus, and a control method of the ultrasound diagnostic apparatus according to an aspect of the present application will be described in the following.

An ultrasound diagnostic apparatus according to an aspect of the present application is an ultrasound diagnostic apparatus configured in a manner connectable to an ultrasound probe and an indicator, the apparatus including: a transmission unit configured to perform transmission processing to drive the ultrasound probe and to transmit an ultrasound to a subject; a reception unit configured to perform reception processing to generate a reception signal based on a reflected ultrasound which is from the subject and is received by the ultrasound probe; a B mode image generation unit configured to generate a B mode image based on the reception signal; a C mode image generation unit configured to generate a first C mode image based on the reception signal; and a display processing unit configured to generate a composite image in which the C mode image is superimposed and displayed on the B mode image and to output the composite image to the indicator, wherein the C mode image generation unit compares the first C mode image data and second C mode image data generated before the first C mode image data, determines whether there is a noise part in the first C mode image data based on the comparison result, eliminates the noise part when there is the noise part in the first C mode image data, and generates a first C mode image based on the first C mode image data from which the noise part is eliminated.

A controller of an ultrasound diagnostic apparatus according to an aspect of the present application is a controller of an ultrasound diagnostic apparatus configured in a manner connectable to an ultrasound probe and an indicator, the controller including: a transmission unit configured to perform transmission processing to drive the ultrasound probe and to transmit an ultrasound to a subject; a reception unit configured to perform reception processing to generate a reception signal based on a reflected ultrasound which is from the subject and is received by the ultrasound probe; a B mode image generation unit configured to generate a B mode image based on the reception signal; a C mode image generation unit configured to generate a first C mode image based on the reception signal; and a display processing unit configured to generate a composite image in which the C mode image is superimposed and displayed on the B mode image and to output the composite image to the indicator, wherein the C mode image generation unit compares the first C mode image data and second C mode image data generated before the first C mode image data, determines whether there is a noise part in the first C mode image data based on the comparison result, eliminates the noise part when there is the noise part in the first C mode image data, and generates a first C mode image based on the first C mode image data from which the noise part is eliminated.

A control method of an ultrasound diagnostic apparatus according to an aspect of the present application is a control method of an ultrasound diagnostic apparatus configured in a manner connectable to an ultrasound probe and an indicator, the method including: a process A to perform transmission processing to drive the ultrasound probe and to transmit an ultrasound to a subject; a process B to perform reception processing to generate a reception signal based on a reflected ultrasound which is from the subject and is received by the ultrasound probe; a process C to generate a B mode image based on the reception signal; a process D to generate a first C mode image based on the reception signal; and a process E to generate a composite image in which the C mode image is superimposed and displayed on the B mode image and to output the composite image to the indicator, wherein the process D includes a process to compare the first C mode image data and second C mode image data generated before the first C mode image data, to determine whether there is a noise part in the first C mode image data based on the comparison result, to eliminate the noise part when there is a noise part in the first C mode image data, and to generate a first C mode image based on the first C mode image data from which the noise part is eliminated.

Hereinafter, an ultrasound diagnostic apparatus, a controller of the ultrasound diagnostic apparatus, and a control method of the ultrasound diagnostic apparatus according to an aspect of an embodiment of the present application will be described with reference to the drawings. However, the scope of the invention is not limited to the illustrated examples.

### First Embodiment

Hereinafter, an ultrasound diagnostic apparatus, a controller of the ultrasound diagnostic apparatus, and a control method of the ultrasound diagnostic apparatus according to the first embodiment will be described with reference to the drawings.

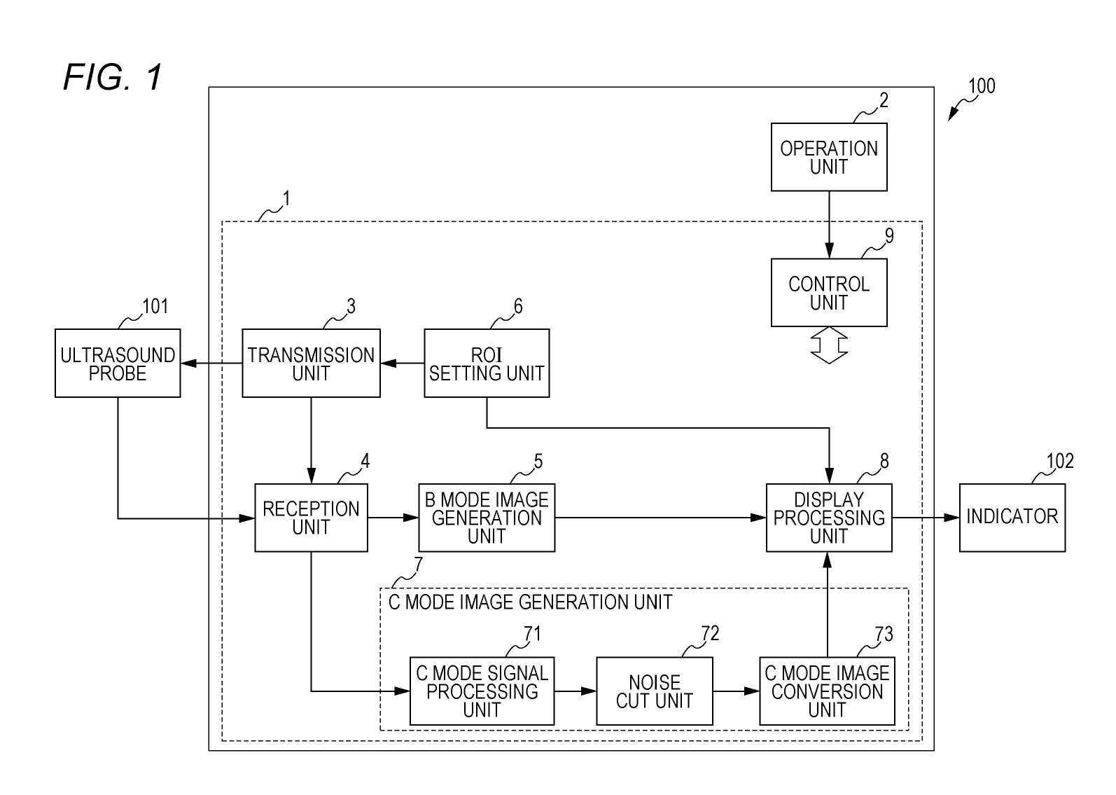

FIG. 1 is a schematic block diagram illustrating a configuration of the ultrasound diagnostic apparatus according to the first embodiment. To the ultrasound diagnostic apparatus 100 in FIG. 1, an ultrasound probe 101 and an indicator 102 are connected.

The ultrasound diagnostic apparatus 100 illustrated in FIG. 1 includes a controller 1 and an operation unit 2. The controller 1 includes a transmission unit 3, a reception unit 4, a B mode image generation unit 5, an ROI setting unit 6, a C mode image generation unit 7, a display processing unit 8, and a control unit 9.

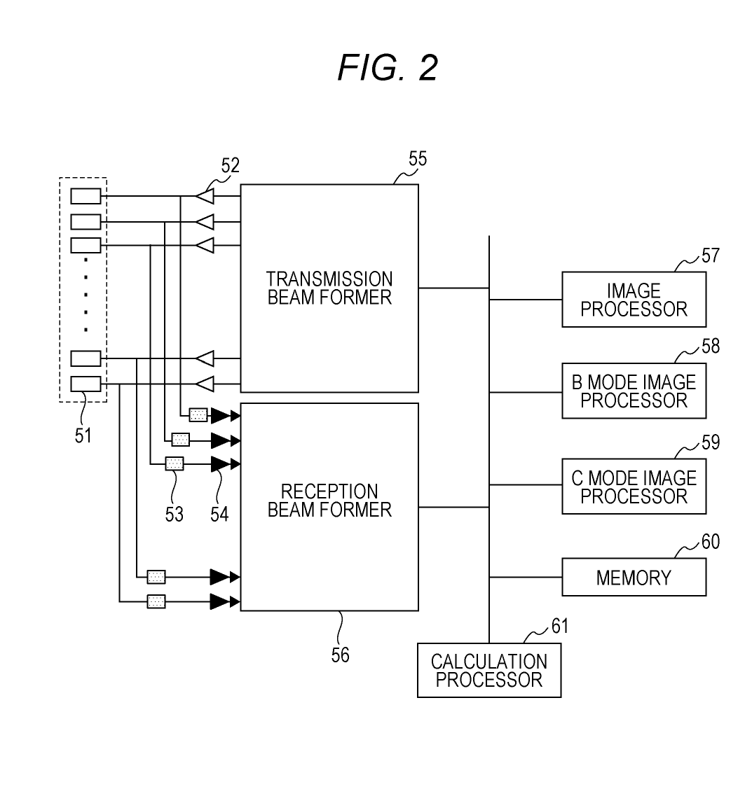

FIG. 2 is a view illustrating a main configuration of hardware of the ultrasound diagnostic apparatus 100. In a standpoint of the hardware, the ultrasound diagnostic apparatus 100 includes, for example, a pulsar 52, an amplifier 53, an A/D converter 54, a transmission beam former 55, a reception beam former 56, an image processor 57, a B mode image processor 58, a C mode image processor 59, a memory 60, and a calculation processor 61. The ultrasound probe 101 includes a plurality of piezoelectric transducing elements 51 to transmit/receive an ultrasound. The number of pulsars 52, amplifiers 53, and A/D converters 54 corresponds to the number of piezoelectric transducing elements 51. The memory 60 stores a program which defines a procedure to realize a function of each element illustrated in FIG. 1 and a program which defines a procedure to control the ultrasound diagnostic apparatus 100, the ultrasound probe 101, and the indicator 102 and to generate and display a B mode image and a C mode image described later by making each element operate in a predetermined procedure. These programs are successively read from the memory 60 and are executed by the calculation processor 61.

Each element illustrated in FIG. 1 includes hardware illustrated in FIG. 2.

The transmission unit 3 includes the pulsar 52 and the transmission beam former 55. The reception unit 4 includes the amplifier 53, the A/D converter 54, and the reception beam former 56. The B mode image generation unit 5 includes the image processor 57 and the B mode image processor 58. The C mode image generation unit 7 includes the C mode image processor 59. The display processing unit 8 includes the image processor 57, the B mode image processor 58, and the C mode image processor 59.

A function of the ROI setting unit 6 is realized by software. Specifically, the programs stored in the memory 60 are executed by the calculation processor 61, and thus, the function of the ROI setting unit 6 is realized. That is, it can be said that the ROI setting unit 6 is configured by the programs.

The above described configuration of the hardware is an example and various modifications can be made thereto. For example, a function of the B mode image generation unit 5 or the C mode image generation unit 7 may be realized by software. Also, functions of the transmission beam former 55 and the reception beam former 56 may be realized by software. A personal computer including the calculation processor 61, the memory 60, and the image processor 57 may be used instead of the hardware.

Also, in respect to each function block of the controller 1, apart or the whole of a function of each function block can be typically realized as an LSI which is an integrated circuit. Each of these may be realized as one chip, or a part or the whole thereof may be realized as one chip. Note that here, the LSI is used but may be referred to as an IC, a system LSI, a super LSI, an ultra LSI depending on a degree of integration.

Also, a method of circuit integration is not limited to the LSI and may be realized by a special circuit or a general processor. A field programmable gate array (FPGA) which can be programed after production of an LSI or a reconfigurable processor in which connection or setting of a circuit cell inside an LSI can be reconfigured after production of the LSI may be used.

Moreover, when circuit integration technique which replaces the LSI appears by progress in semiconductor technique or by different technique derived therefrom, integration of a function block may be performed by using the technique in all reason.

As described, the ultrasound probe 101 includes a plurality of piezoelectric transducing elements 51 arrayed in one-dimensional direction. Each of the piezoelectric transducing elements 51 converts a transmission electric signal, which is from the transmission unit 3 described later, into an ultrasound and generates an ultrasound beam. Thus, by arranging the ultrasound probe 101 on a surface of a subject which is an object to be measured, an operator can emit an ultrasound beam to the inside of the subject. Then, the ultrasound probe 101 receives a reflected ultrasound from the inside of the subject. The ultrasound probe 101 converts, with the plurality of piezoelectric transducing elements 51, the reflected ultrasound into a reception electric signal and supplies the converted signal to the reception unit 4 described later.

Note that in the first embodiment, the ultrasound probe 101 will be described with the ultrasound probe 101 in which the plurality of piezoelectric transducing elements 51 is arrayed in one-dimensional direction as an example. But the example is not a limitation. For example, an ultrasound probe 101 in which the plurality of piezoelectric transducing elements 51 is arranged two-dimensionally or an ultrasound probe 101 in which the plurality of piezoelectric transducing elements 51 arrayed in one-dimensional direction rocks can be used. Also, based on control by the control unit 9, by selecting a piezoelectric transducing element 51 used by the ultrasound probe 101 and changing timing to give voltage to the piezoelectric transducing element 51 and a value of the voltage respectively, the transmission unit 3 can control an emitted position or an emitted direction of an ultrasound beam transmitted by the ultrasound probe 101.

Also, the ultrasound probe 101 may include a part of a function of the transmission unit 3 or the reception unit 4 which will be described later. For example, based on a control signal (hereinafter, referred to as “transmission signal”) which is output from the transmission unit 3 and is to generate a transmission electric signal, the ultrasound probe 101 generates a transmission electric signal in the ultrasound probe 101 and converts the transmission signal into an ultrasound by the piezoelectric transducing elements 51 and a received reflected ultrasound into a reception electric signal. A reception signal described later is generated in the ultrasound probe 101 based on the reception electric signal.

Moreover, the ultrasound probe 101 is generally connected to the ultrasound diagnostic apparatus 100 electrically through a cable but is not limited thereto. For example, the ultrasound probe may receive/transmit a transmission signal or a reception signal from/to the ultrasound diagnostic apparatus 100 by wireless communication. In such a case, each of the ultrasound diagnostic apparatus 100 and the ultrasound probe 101 includes a communication unit which can perform wireless communication.

The indicator 102 is so-called a monitor to display an image output from the ultrasound diagnostic apparatus 100 (display processing unit 8 described later). Note that in the first embodiment, the indicator 102 is connected to the ultrasound diagnostic apparatus 100. However, for example, in a case of a touch panel type ultrasound diagnostic apparatus in which the indicator 102 and the operation unit 2 described later are integrally configured and an operation of the operation unit 2 is performed by a touch operation on the indicator 102, the ultrasound diagnostic apparatus 100 and the indicator 102 are formed integrally. However, in the present application, even in a case where the ultrasound diagnostic apparatus 100 and the indicator 102 are configured integrally, it is assumed that “the indicator 102 is connected to the ultrasound diagnostic apparatus 100”.

The operation unit 2 receives an input from an operator and outputs a command based on the input from the operator to the ultrasound diagnostic apparatus 100, specifically, to the control unit 9 of the controller 1. The operation unit 2 has a function with which the operator can select a mode whether to display only a B mode image (hereinafter, referred to as “B mode”) or to superimpose and display a C mode image on the B mode image (hereinafter, referred to as “C mode”). Also, the operation unit 2 has a function with which the operator specifies a position of the ROI on the B mode image on which position the C mode image is displayed.

The transmission unit 3 at least performs transmission processing to generate a transmission signal and to make the ultrasound probe 101 transmit an ultrasound beam. For example, the transmission unit 3 performs transmission processing to generate a transmission signal to transmit an ultrasound beam from the ultrasound probe 101 including the piezoelectric transducing elements 51 and supplies a transmission electric signal at high voltage, which signal is generated at predetermined timing, to the ultrasound probe 101 based on the transmission signal. Thus, the piezoelectric transducing elements 51 of the ultrasound probe 101 are driven. Accordingly, by converting the transmission electric signal into an ultrasound, the ultrasound probe 101 can emit an ultrasound beam to a subject which is an object to be measured.

In a case of displaying the C mode image, the transmission unit 3 performs transmission processing to display the C mode image in addition to transmission processing to display the B mode image. For example, after a transmission electric signal to display the B mode image is supplied, a transmission electric signal to display the C mode image is supplied for a plurality of times in a direction of the ROI which is set by the ROI setting unit 6. Also, the transmission unit 3 specifies additional information of transmission processing for a B mode image or of transmission processing for a C mode image during the transmission processing and supplies the additional information to the reception unit 4.

Note that the transmission processing of the transmission unit 3 to display the C mode image is publicly-known technique. The above described transmission processing is just an example and is not a limitation.

The reception unit 4 at least performs reception processing to generate a reception signal based on a reflected ultrasound. For example, the reception unit 4 receives a reflected ultrasound with the ultrasound probe 101 and amplifies a reception electric signal converted based on the reflected ultrasound and performs an A/D conversion thereon, whereby a reception signal is generated. Then, by execution of the transmission processing by the transmission unit 3 and execution of the reception processing by the reception unit 4, a plurality of reception signals corresponding to one image frame is acquired. By repeating the execution continuously, the reception unit 4 acquires a plurality of reception signals corresponding to a plurality of image frames.

The reception unit 4 acquires additional information from the transmission unit 3. In a case where the acquired additional information is additional information for a B mode image, the reception unit 4 supplies the reception signal to the B mode image generation unit 5 and in a case where the acquired additional information is additional information for a C mode image, the reception unit 4 supplies the reception signal to the C mode image generation unit 7. Hereinafter, a reception signal for B mode image generation will be referred to as a “B mode reception signal” and a reception signal for C mode image generation will be referred to as a “C mode reception signal”.

Note that in the first embodiment, the reception unit 4 classifies whether a generated reception signal is for a B mode image or a C mode image and supplies the signal to each block but this example is not a limitation. For example, a generated reception signal may be classified in each of the B mode image generation unit 5 and the C mode image generation unit 7.

The B mode image generation unit 5 includes a similar structure with a general ultrasound diagnostic apparatus. The B mode image generation unit 5 mainly analyzes amplitude of the B mode reception signal and generates data which is an imaged inner structure of a subject (hereinafter, referred to as “B mode image data”). The B mode image data is data to be displayed on the indicator 102 and is an image signal which is converted into a luminance signal mainly according to signal strength of a reception signal and on which coordinate transformation is performed to make the luminance signal correspond to an orthogonal coordinate system. The B mode image data generated in the B mode image generation unit 5 is supplied to the display processing unit 8.

The ROI setting unit 6 sets the ROI in an intended position on the B mode image which position is specified by an operation of the operation unit 2 by an operator. Then, the ROI setting unit 6 supplies, to the transmission unit 3 and the display processing unit 8, information related to the ROI set in the intended position on the B mode image. By using the information related to the ROI, the transmission unit 3 can perform transmission processing corresponding to a C mode on a subject within the specified range of the ROI.

The C mode image generation unit 7 generates a C mode image based on the C mode reception signal acquired in the reception unit 4. Specifically, the C mode image generation unit 7 includes the C mode signal processing unit 71, the noise cut unit 72, and a C mode image conversion unit 73. These units respectively execute the following functions.

By performing orthogonal detection, the C mode signal processing unit 71 calculates a phase difference between the acquired C mode reception signal and a reference signal and acquires a complex Doppler signal. Here, the reference signal is a signal multiplied by the C mode reception signal in the orthogonal detection. Then, the C mode signal processing unit 71 acquires a complex Doppler signal sequence by repeating transmission/reception of an ultrasound.

In the acquired C mode reception signal, a signal related to a blood vessel wall or a biological tissue is included in addition to bloodstream information. Thus, by an MTI filter included in the C mode signal processing unit 71, a complex Doppler signal which has a radio-frequency wave and small amplitude and is a bloodstream component is extracted from the complex Doppler signal sequence. Then, by performing processing such as autocorrelation calculation on the extracted complex Doppler signal sequence, a signal indicating a state of bloodstream such as speed, power, or variance of the bloodstream (hereinafter, referred to as “bloodstream signal”) is calculated.

The noise cut unit 72 eliminates a clutter noise from the extracted bloodstream signal.

Since being generated due to movement of a tissue (including relative movement accompanying movement of ultrasound probe 101), the clutter noise generally includes a relatively high frequency component compared to a signal component of a static tissue.

Thus, since the MTI filter included in the C mode signal processing unit 71 cannot eliminate a clutter noise included in the complex Doppler signal sequence adequately, the ultrasound diagnostic apparatus 100 of the present application includes the noise cut unit 72.

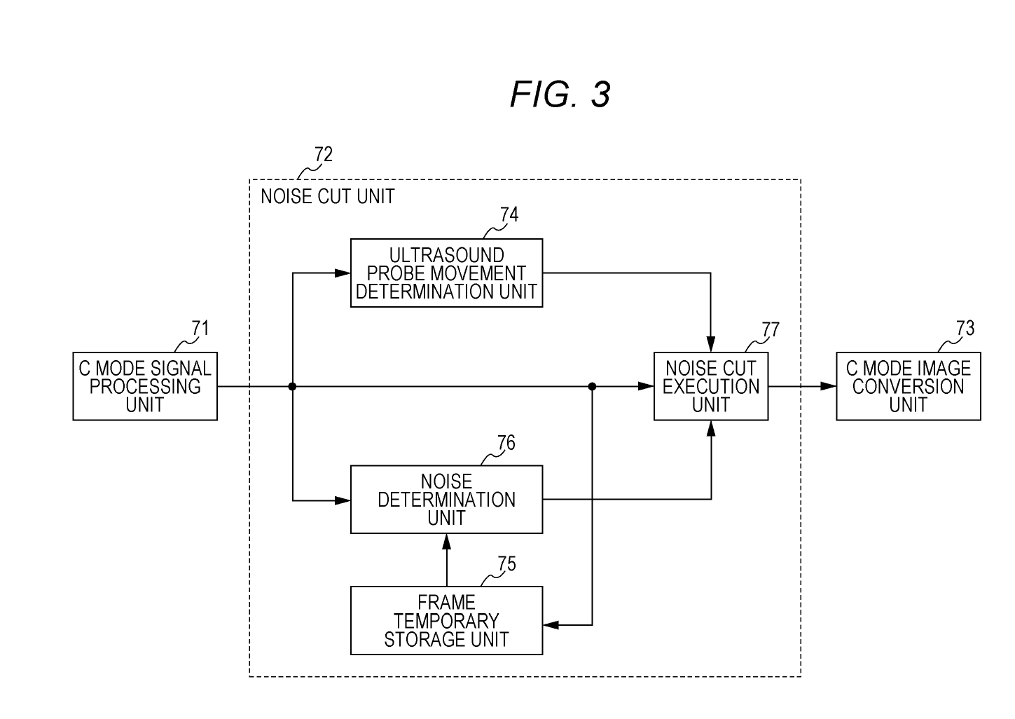

As illustrated in FIG. 3, the noise cut unit 72 includes the ultrasound probe movement determination unit 74, the frame temporary storage unit 75, the noise determination unit 76, and the noise cut execution unit 77 and executes the following functions.

The ultrasound probe movement determination unit 74 determines whether there is movement of the ultrasound probe 101. The ultrasound probe movement determination unit 74 includes a predetermined threshold in advance and determines, by using the predetermined threshold, whether there is movement of the ultrasound probe 101 based on a movement determination quantity of the ultrasound probe 101. Note that the movement determination quantity of the ultrasound probe 101 is information indicating whether there is movement in the ultrasound probe 101, and is a rate of movement or acceleration in a unit time of the ultrasound probe 101 or different information which changes along with the movement of the ultrasound probe.

When it is determined by the ultrasound probe movement determination unit 74 that there is movement of the ultrasound probe 101, a noise is eliminated by the noise determination unit 76 and the noise cut execution unit 77 which will be described later. When it is determined that there is no movement, processing by the noise determination unit 76 and the noise cut execution unit 77 is skipped.

Note that in the first embodiment, since a configuration to eliminate a clutter noise which appears prominently in a case of performing an ultrasound image diagnosis while moving the ultrasound probe 101 has been described, the ultrasound probe movement determination unit 74 is included. However, specifically when an ultrasound image diagnosis is performed with the ultrasound probe 101 being fixed, the present function block may not be included.

In respect to a complex Doppler signal sequence to which the MTI filter is not yet applied, the ultrasound probe movement determination unit 74 detects a Doppler shift frequency by a fast Fourier transform (FFT) analysis and calculates a spread quantity of a Doppler shift from the Doppler shift frequency. Then, based on the spread quantity, the ultrasound probe movement determination unit 74 detects speed in a direction orthogonal to a progress direction of the ultrasound, and thus, a rate of movement of the ultrasound probe 101 is calculated. The ultrasound probe movement determination unit 74 performs threshold processing on the calculated rate of movement of the ultrasound probe 101 by using a predetermined threshold stored in advance and determines whether there is movement of the ultrasound probe 101.

Note that a method to calculate a rate of movement of an ultrasound probe based on a complex Doppler signal sequence is based on technique described in JP 2000-107187 A.

Also, in JP 2000-107187 A, a rate of movement of an ultrasound probe 101 is calculated based on a Doppler shift frequency but the present application is not limited thereto. For example, in acquiring a C mode image of one image frame, transmission/reception of an ultrasound to/from a subject is performed for a plurality of times. However, to determine whether there is movement of the ultrasound probe 101, average speed may be compared with a predetermined threshold based on a bloodstream signal (such as average speed information) calculated from a complex Doppler signal sequence acquired by the plurality of times of transmission/reception. That is, in the present application, there is not a specific limitation in a configuration, as long as it is determined based on the C mode reception signal whether there is movement of the ultrasound probe 101.

Moreover, calculation of a movement determination quantity of the ultrasound probe 101 is not limited to a method based on the C mode reception signal. For example, the calculation may be performed by a sensor embedded in the ultrasound probe 101 or provided separately to the outside of the ultrasound probe 101. A sensor used in this case is not particularly limited as long as the movement determination quantity can be calculated. For example, an acceleration sensor, a gyro sensor, or an optical sensor can be used. In this case, instead of performing movement determination of the ultrasound probe 101 based on an extracted complex Doppler signal sequence of bloodstream component as illustrated in FIG. 3, the ultrasound probe movement determination unit 74 performs movement determination based on output information from the sensor provided inside or outside the ultrasound probe 101.

The frame temporary storage unit 75 successively stores a bloodstream signal of an image frame supplied from the C mode signal processing unit 71 and temporarily stores a bloodstream signal of a predetermined number of image frames, the number being one or more. Every time a bloodstream signal of a new image frame is supplied from the C mode signal processing unit 71, the frame temporary storage unit 75 eliminates a bloodstream signal related to the oldest image frame and stores a bloodstream signal of a newly supplied image frame.

Then, the frame temporary storage unit 75 supplies, to the noise determination unit 76, a bloodstream signal of a predetermined number of image frames, the number being one or more, generated before an image frame which becomes an object of determination in the noise determination unit 76 which will be described later.

The noise determination unit 76 compares a bloodstream signal of the image frame supplied from the C mode signal processing unit 71 with a bloodstream signal of an image frame supplied from the frame temporary storage unit 75 and determines a noise part in the image frame.

Based on output information from the noise determination unit 76, the noise cut execution unit 77 eliminates, from an object frame, a bloodstream signal in the noise part.

Specifically, the noise cut execution unit 77 sets, as an object of noise elimination, an image frame in which it is determined by the ultrasound probe movement determination unit 74 that there is movement of the ultrasound probe 101. The noise cut execution unit 77 performs processing not to display, as the bloodstream information, a part determined as a noise in the image frame by the noise determination unit 76.

Note that as described above, in the first embodiment, specifically a case of diagnosing with an ultrasound image while moving the ultrasound probe 101 such as a case of an ultrasound diagnosis of a finger or the like of a patient with rheumatoid arthritis is assumed. Also, in order to eliminate a noise accurately, an image frame on which processing of noise elimination is performed is classified based on a determination result by the ultrasound probe movement determination unit 74. However, this example is not a limitation. That is, in a case of diagnosing with the ultrasound probe 101 being fixed to a surface of a subject, the ultrasound probe movement determination unit 74 may not be included (that is, image frame to be object of noise elimination depending on movement of ultrasound probe 101 is not classified).

The C mode image conversion unit 73 generates a C mode image in respect to a bloodstream signal in an image frame processed in the noise cut unit 72.

The display processing unit 8 constructs display image data to be displayed on the indicator 102 and performs processing to display the display image data on the indicator 102. Specifically, when the B mode is selected, processing to display a B mode image, which is generated in the B mode image generation unit 5, as an ultrasound image in display screen data is performed. Also, when the C mode is selected, processing to generate composite image data, in which a C mode image generated in the C mode image generation unit 7 is superimposed on an position of the ROI selected on the B mode image generated in the B mode image generation unit 5, and to display the data as an ultrasound image in display screen data is performed.

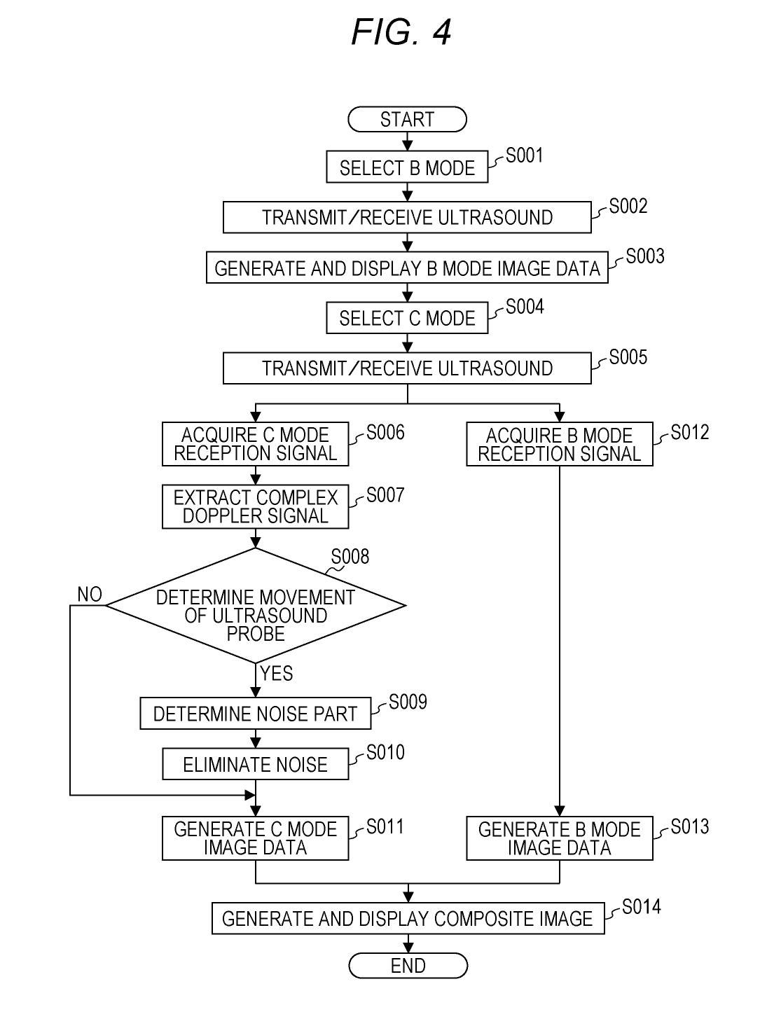

A detail operation of the ultrasound diagnostic apparatus 100 including the above described configuration will be described with reference to an operation flowchart in FIG. 4 in accordance with an operation of an operator. Here, an operation related to processing of noise elimination of a C mode image (that is, generation of C mode image) will be mainly described. Other operations will be omitted since being similar to those of a general ultrasound diagnostic apparatus.

In step 1 (S001), an operator selects a B mode by operating the operation unit 2 and performs various kinds of setting to acquire a B mode image.

In step 2 (S002), by performing transmission processing corresponding to the B mode based on the setting of the operation unit 2, the transmission unit 3 transmits an ultrasound to the inside of a subject through the ultrasound probe 101. Then, the reception unit 4 receives the reflected ultrasound through the ultrasound probe 101 and performs reception processing to generate a B mode reception signal. By repeatedly performing the transmission processing and the reception processing, a plurality of reception signals for each image frame is generated.

In step 3 (S003), the B mode image generation unit 5 generates B mode image data by performing, on each image frame, conversion into a luminance signal corresponding to signal strength of the B mode reception signal and by performing coordinate transformation in such a manner that the luminance signal corresponds to an orthogonal coordinate system. Then, based on the B mode image data generated in the B mode image generation unit 5, the display processing unit 8 generates display screen data and outputs the generated data to the indicator 102. Accordingly, an image including the generated B mode image is displayed on the indicator 102.

In step 4 (S004), the operator selects a C mode with the operation unit 2. Then, while checking the B mode image displayed on the indicator 102, the operator operates the operation unit 2 and sets an ROI in the B mode image displayed on the indicator 102, whereby a region to display the C mode image is specified.

In step 5 (S005), to acquire a reception signal of one image frame, the transmission unit 3 performs transmission processing to generate a B mode image and transmission processing to generate a C mode image in the ROI. Then, by the transmission processing in the transmission unit 3, the reception unit 4 generates a B mode reception signal and a C mode reception signal in one image frame and gives additional information thereto.

In step 6 (S006), based on the additional information given by the reception unit 4, the C mode signal processing unit 71 acquires a C mode reception signal.

In step 7 (S007), by performing orthogonal detection in one image frame, the C mode signal processing unit 71 detects a phase difference between the acquired C mode reception signal and a reference signal and acquires a complex Doppler signal. Then, the C mode signal processing unit 71 acquires a complex Doppler signal sequence by repeating transmission/reception of an ultrasound and extracts a complex Doppler signal, which has a radio-frequency wave and small amplitude and is a bloodstream component, with the MTI filter from the complex Doppler signal sequence.

In step 8 (S008), when the ultrasound probe movement determination unit 74 corresponds to the image frame which is the object of the processing in step 7 (S007), it is determined with a predetermined threshold whether there is movement of the ultrasound probe 101. In respect to an image frame in which it is determined that there is movement of the ultrasound probe (“Yes” in FIG. 4), a step goes to step 9 (S009) and in respect to an image frame in which it is determined that there is no movement of the ultrasound probe (“No” in FIG. 4), a step goes to step 11 (S011).

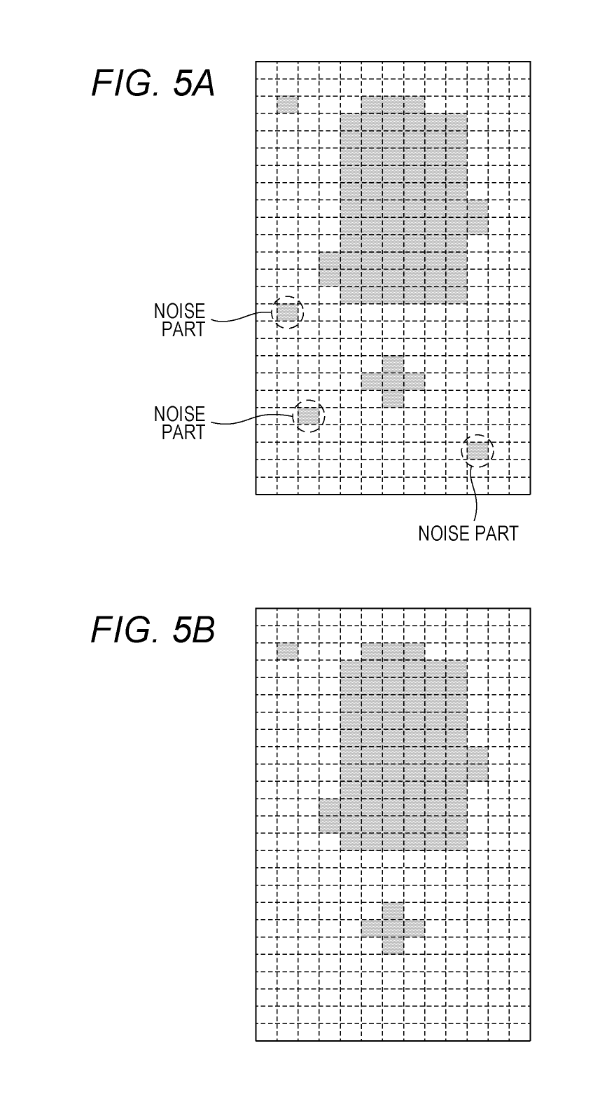

In step 9 (S009), the noise determination unit 76 determines a noise part by comparing a complex Doppler signal of an image frame, which is an object of determination, with that of an image frame generated before the image frame, which is the object of determination, and stored in the frame temporary storage unit 75. This point will be described in detail with reference to FIG. 5A and FIG. 5B.

FIG. 5A is a view illustrating an image frame which is an object of determination in the noise determination unit 76. FIG. 5B is a view illustrating an image frame stored in the frame temporary storage unit 75. In FIG. 5A and FIG. 5B, a shaded part indicates that there is bloodstream information. Here, in a case where speed/power of a bloodstream signal exceeds a predetermined threshold, it is said that there is bloodstream information.

The image frame in FIG. 5B needs to be an image frame generated before the image frame which is the object of determination and is preferably an image frame generated right before the image frame which is the object of determination.

The noise determination unit 76 compares the image frame, which is the object of determination, with an image frame stored in the frame temporary storage unit 75 and extracts a noise part. For example, in an example in FIG. 5A and FIG. 5B, there is bloodstream information in a part surrounded by a circle with a broken line in FIG. 5A but there is no bloodstream information in the same part in the image frame stored in the frame temporary storage unit 75 in FIG. 5B, thus the noise determination unit 76 determines the part surrounded by a circle with a broken line in FIG. 5A as a noise part.

In step 10 (S010), based on a result of the noise determination in step 9 (S009), the noise cut execution unit 77 performs processing of noise elimination.

That is, when it is determined that there is a noise in step 9 (S009) as illustrated in FIG. 5A and FIG. 5B, the noise cut execution unit 77 eliminates a part determined as the noise. Then, a step goes to step 11 (S011).

In step 11 (S011), the C mode image conversion unit 73 generates a C mode image based on a complex Doppler signal in the image frame.

In step 12 (S012), the B mode image generation unit 5 acquires a B mode reception signal based on the additional information given by the reception unit 4.

In step 13 (S013), the B mode image generation unit 5 generates B mode image data by performing, on each image frame, conversion into a luminance signal corresponding to signal strength of the B mode reception signal and by performing coordinate transformation in such a manner that the luminance signal corresponds to an orthogonal coordinate system.

In step 14 (S014), the display processing unit 8 generates composite image data in which the C mode image data generated in step 11 (S011) is superimposed on a position, in which the ROI is set, on the B mode image data generated in step (S013). Then, the display processing unit 8 generates display screen data including the composite image data and outputs the generated data to the indicator 102, whereby a generated image including the B mode image and the C mode image is displayed on the indicator 102.

As described, according to the ultrasound diagnostic apparatus, the controller of the ultrasound diagnostic apparatus, and the control method of the ultrasound diagnostic apparatus described in the first embodiment, a noise image in a C mode image can be effectively controlled.

Specifically, in a case where a noise is eliminated only when determination of movement of an ultrasound probe is performed and it is determined that there is movement of the ultrasound probe, a clutter noise generated by the movement of the ultrasound probe can be eliminated accurately. When diagnosis is performed with the ultrasound probe being fixed to a surface of a subject, it can be prevented that bloodstream which appears on and disappears from an image frame due to pulsatility is eliminated as a noise by mistake.

According to the ultrasound diagnostic apparatus, the controller of the ultrasound diagnostic apparatus, and the control method of the ultrasound diagnostic apparatus according to an aspect of the present application, generation of a noise image in a C mode image can be controlled effectively. Specifically, an aspect of the present application is useful for diagnosis which is performed while an ultrasound probe is moved and in which a clutter noise is likely to be generated, such as an ultrasound image diagnosis of rheumatoid arthritis.

Although the present invention has been described and illustrated in detail, it is clearly understood that the same is by way of illustration and example only and is not to be taken by way of limitation, the scope of the present invention being interpreted by terms of the appended claims.

What is claimed is:

1. An ultrasound diagnostic apparatus configured in a manner connectable to an ultrasound probe and an indicator, comprising:
a transmission unit configured to perform transmission processing to drive the ultrasound probe and to transmit an ultrasound to a subject;
a reception unit configured to perform reception processing to generate a reception signal based on a reflected ultrasound which is from the subject and is received by the ultrasound probe;
a B mode image generation unit configured to generate a B mode image based on the reception signal;
a C mode image generation unit configured to generate a first C mode image based on the reception signal; and
a display processing unit configured to generate a composite image in which the C mode image is superimposed and displayed on the B mode image and to output the composite image to the indicator,
wherein the C mode image generation unit compares the first C mode image data and second C mode image data generated before the first C mode image data, determines whether there is a noise part in the first C mode image data based on the comparison result, eliminates the noise part when there is the noise part in the first C mode image data, and generates a first C mode image based on the first C mode image data from which the noise part is eliminated.

2. The ultrasound diagnostic apparatus according to claim 1, wherein the C mode image generation unit compares the first C mode image data and the second C mode image data generated before the first C mode image data and determines, as the noise part, a part of the first C mode image data in which part there is no bloodstream information in the second C mode image data and there is bloodstream information in the first C mode image data.

3. The ultrasound diagnostic apparatus according to claim 1, wherein the second C mode image data is C mode image data generated right before the first C mode image data.

4. The ultrasound diagnostic apparatus according to claim 1, further comprising an ultrasound probe movement determination unit configured to determine whether there is movement of the ultrasound probe,
wherein the C mode image generation unit performs processing, which is to eliminate the noise part, on the first C mode image data in which it is determined by the ultrasound probe movement determination unit that there is movement of the ultrasound probe.

5. The ultrasound diagnostic apparatus according to claim 4, wherein based on a complex Doppler signal sequence extracted based on the reception signal or a bloodstream signal calculated from the complex Doppler signal sequence, the ultrasound probe movement determination unit determines whether there is movement of the ultrasound probe.

6. The ultrasound diagnostic apparatus according to claim 4, wherein based on output information from a sensor which is provided inside or outside the ultrasound probe and is configured to determine a movement determination quantity, the ultrasound probe movement determination unit determines whether there is movement of the ultrasound probe.

7. A controller of an ultrasound diagnostic apparatus configured in a manner connectable to an ultrasound probe and an indicator, comprising:
a transmission unit configured to perform transmission processing to drive the ultrasound probe and to transmit an ultrasound to a subject;
a reception unit configured to perform reception processing to generate a reception signal based on a reflected ultrasound which is from the subject and is received by the ultrasound probe;
a B mode image generation unit configured to generate a B mode image based on the reception signal;
a C mode image generation unit configured to generate a first C mode image based on the reception signal; and
a display processing unit configured to generate a composite image in which the C mode image is superimposed and displayed on the B mode image and to output the composite image to the indicator,
wherein the C mode image generation unit compares the first C mode image data and second C mode image data generated before the first C mode image data, determines whether there is a noise part in the first C mode image data based on the comparison result, eliminates the noise part when there is the noise part in the first C mode image data, and generates a first C mode image based on the first C mode image data from which the noise part is eliminated.

8. A control method of an ultrasound diagnostic apparatus configured in a manner connectable to an ultrasound probe and an indicator, the method comprising:
a process A to perform transmission processing to drive the ultrasound probe and to transmit an ultrasound to a subject;
a process B to perform reception processing to generate a reception signal based on a reflected ultrasound which is from the subject and is received by the ultrasound probe;
a process C to generate a B mode image based on the reception signal;
a process D to generate a first C mode image based on the reception signal; and
a process E to generate a composite image in which the C mode image is superimposed and displayed on the B mode image and to output the composite image to the indicator,
wherein the process D includes a process to compare the first C mode image data and second C mode image data generated before the first C mode image data, to determine whether there is a noise part in the first C mode image data based on the comparison result, to eliminate the noise part when there is the noise part in the first C mode image data, and to generate a first C mode image based on the first C mode image data from which the noise part is eliminated.
